(19) United States
(12) Patent Application Publication
(10) Pub. No.: US 2011/0223177 A1
(43) Pub. Date: Sep. 15, 2011

(54) TREATMENT OF FIBROTIC EYE DISORDERS

(75) Inventors: Ian Michael Wormstone, Norfolk (GB); Lucy Dawes, Norfolk (GB); Julie Ann Eldred, Norfolk (GB); Lixin Wang, Norfolk (GB)

(73) Assignee: UNIVERSITY OF EAST ANGLIA, (GB)

(21) Appl. No.: 13/063,132

(22) PCT Filed: Sep. 9, 2009
(86) PCT No.: PCT/GB2009/051146

§ 371 Date: May 24, 2011

Related U.S. Application Data
(60) Provisional application No. 61/095,547, filed on Sep. 9, 2008.

(51) Int. Cl. A61K 39/395; C07D 243/08; A61K 31/551; A61P 27/02; C07K 7/06; A61K 38/08; C07C 311/18; A61K 31/18; C07H 21/02; A61K 31/713; C07K 16/40

(52) U.S. Cl. 424/146.1; 540/575; 514/218; 530/328; 514/20.8; 564/84; 514/602; 536/24.5; 514/44 A; 530/389.1; 424/172.1; 530/388.26

(57) ABSTRACT

Inhibitors of myosin activity are used to treat or prevent a fibrotic disorder of the eye, for example posterior capsule opacification (PCO).

### FIELD OF THE INVENTION

The present invention relates to the treatment of fibrotic eye disorders, for example TGF-β-mediated fibrotic (including tissue-contraction) disorders of the lens, capsular bag, cornea, conjunctiva, sclera and other tissues or structures of the eye, particularly but not exclusively fibrotic complications following eye surgery in humans.

### BACKGROUND OF THE INVENTION

Fibrotic disorders of the eye are common complications arising from surgical treatment of disorders including glaucoma, pterygia and cataract. Many of the underlying mechanisms giving rise to these fibrotic disorders are likely to share common pathways. Further details and discussion of fibrotic complications of glaucoma surgery, cataract or other ocular lens replacement surgery and pterygia surgery are found respectively in Cordeiro, M. F., Prog. Retin. Eye Res. (2002) 21, pages 75-89, Wormstone, I. M., Exp. Eye Res. (2002) 74, pages 337-347, and Di Girolamo, N., et al., Prog. Retin. Eye Res. (March 2004), 23(2), pages 195-228, the contents of all of which are incorporated herein by reference. For example, PCO is the most common fibrotic complication following cataract or other ocular lens replacement surgery. This condition is caused by regrowth of the lens epithelial cells which, despite the surgeon's best efforts, typically remain on the anterior capsule after the surgery. The cellular regrowth typically invades denuded surfaces of the anterior capsule, the implanted intraocular lens and the previously cell-free posterior capsule. The epithelial cells on the posterior capsule surface give rise to contraction of the tissue matrix, leading to opacification of the posterior capsule and reduction in vision quality.

Transforming growth factor β (TGF-β) has been implicated in a number of fibrotic disorders of the lens, capsular bag, cornea, conjunctiva, sclera and other tissues or structures of the eye. For example, TGF-β has been shown to induce anterior subcapsular cataract (ASC) in a rat lens culture model (Gordon-Thomas, C. et al., Invest. Ophthalmol. Vis. Sci. (1998) 39, pages 1399-1409; Hales, A. M. et al., Invest. Ophthalmol. Vis. Sci. (1995) 36, pages 1709-1713). TGF-β has also been implicated as a causative factor in PCO (Saika, S. et al., Graefes. Arch. Clin. Exp. Ophthalmol. (2000) 238, pages 283-293; Wormstone, I. M. et al., Invest. Ophthalmol. Vis. (2002) 43, pages 2301-2308). It has been reported that, after trauma (e.g. surgery), active levels of all TGF-β isoforms can be elevated (Ohta, K. et al., Invest. Ophthalmol. Vis. Sci. (2000) 41, pages 2591-2599). The contents of all of these cited publications are incorporated herein by reference.

Dawes, L. J. et al., Invest. Ophthalmol. Vis. Sci. (February 2008), 49 (2), pages 650-661, the contents of which are incorporated herein by reference, reported that—contrary to previous belief—TGF-β-induced tissue matrix contraction, for example in PCO, appears not to be the result of transdifferentiation of the regrown lens epithelial cells to myofibroblasts, although transdifferentiation does appear to occur in response to the TGF-β isoforms. However, the evidence showed that directed inhibition of markers of transdifferentiation, such as the interaction of fibronectin with its receptor and the activity of α-smooth muscle actin (α-SMA), failed to prevent contraction and indeed could promote contraction. From this it appears that the role of TGF-β in inducing tissue matrix contraction in fibrotic disorders of the lens and surrounding structures, which is one of the major causative factors in the impairment of vision in those suffering from these disorders, may result from an alternative mechanism.

The Dawes et al. publication summarises (page 659, second column) disparate data concerning certain roles of myosin in (myo)fibroblastic and smooth muscle cells. However, the publication by its primary data concludes that the process of transdifferentiation resulting in myofibroblasts (arising from lens epithelial cells) is not directly responsible for matrix contraction in fibrotic eye disorders such as PCO (page 656, first column), and α-SMA is also shown to be not involved in the matrix contraction (page 657, second column). The publication does not provide the reader with enough data to conclude that myosin has a role in a fibrotic disorder of the eye, and therefore does not suggest that affecting myosin activity may have a role in the treatment or prevention of such disorders.

Moreover, in countries which provide inventors with a grace period in respect of the prior art status of their own publications, the Dawes et al. publication is not prior art against the present invention.

The present invention is based on our surprising finding that inhibitors of activated myosin can suppress TGFβ-induced matrix contraction and thus provide a treatment or prevention of a fibrotic disorder of the eye.

### BRIEF DESCRIPTION OF THE INVENTION

In a first aspect, the present invention provides the use, in the treatment or prevention of a fibrotic disorder of the eye, or in the preparation of a medicament therefor, of an active agent comprising an inhibitor of myosin activity.

In a second aspect, the present invention provides an active agent comprising an inhibitor of myosin activity, for use in the treatment or prevention of a fibrotic disorder of the eye.

In a third aspect, the present invention provides a composition, for example a pharmaceutical composition (medicament) comprising an effective amount of an active agent comprising an inhibitor of myosin activity, and one or more physiologically compatible carrier, diluent or excipient, for use in the treatment or prevention of a fibrotic disorder of the eye.

A particular disorder to be treated or prevented is a TGF-β mediated disorder of the lens, capsular bag, cornea, conjunctiva, sclera or other tissue or structure of the eye, for example the posterior lens capsule of the eye. Examples of disorders to be treated or prevented according to the present invention are fibrotic complications of surgery, for example glaucoma surgery (e.g. glaucoma filtration surgery), cataract surgery or other ocular lens replacement surgery, or pterygia surgery, particularly PCO following cataract surgery.

In the various aspects of the present invention, in one embodiment the active agent may, if desired, further comprise at least one other component of the same and/or a different type for treating or preventing a fibrotic disorder of the eye.

The composition may, if desired, contain one or more additional ingredients, which may include effective amounts of one or more components having other physiological activity, suitable amounts of one or more physiologically inert ingredients, or any mixture or combination thereof.

Alternatively, such additional ingredients may be incorporated into other compositions (medicaments), optionally including one or more physiologically compatible carrier, diluent or excipient, for administration separately from the composition according to the present invention, which administration may be prior to, simultaneously with or after the administration of the composition according to the present invention.

The composition according to the present invention may, if desired, consist essentially of the active agent and, if present, the one or more physiologically compatible carrier, diluent or excipient. In this embodiment the active agent may suitably consist essentially or the inhibitor of myosin activity.

The composition according to the present invention may, if desired, consist of the active agent and, if present, the one or more physiologically compatible carrier, diluent or excipient. In this embodiment the active agent may suitably consist of the inhibitor of myosin activity.

The expressions “inhibitor of myosin activity” and “myosin activity inhibitor”, which are interchangeable, used herein include all agents which have the physiological effect of inhibiting the activity or activation of myosin in a subject or the availability of activated myosin for further biochemical reactions, and therefore include a direct-acting inhibitor of the activation (phosphorylation) of myosin in the subject, a direct-acting inhibitor of activated (phosphorylated) myosin in a subject, an indirect-acting inhibitor of the activation (phosphorylation) of myosin in the subject and an indirect-acting inhibitor of activated (phosphorylated) myosin in a subject. The expression “indirect-acting” used herein means that the action is on another substance or group of substances which causes inhibition of the activity or activation of myosin or inhibition of the availability of activated myosin in the subject; when used in connection with other agents the expression shall be understood analogously. An indirect inhibitor of myosin activity includes an agent such as a direct or indirect inhibitor of a myosin activator and a direct or indirect activator of a direct inhibitor of myosin activity. The expression “myosin activator” used herein includes any agent involved in and contributing to the process of activating myosin. The expressions “inhibition of myosin activity”, “myosin activity inhibition” and the like shall be understood analogously.

### DETAILED DESCRIPTION OF THE INVENTION

### Myosin Activity Inhibitor

In the following discussion, the conventional three- and single-letter codes for amino acids are used for naming peptides.

The myosin activity inhibitor may be a specific or non-specific inhibitor of myosin activity.

Suitable inhibitors of myosin activity for use in the present invention include, for example, myosin light chain kinase inhibitors (MLCK inhibitors) such as (5-iodonaphthalene-1-sulfonyl)homopiperazine (ML7), 1-(5-chloronaphthalene-1-sulfonyl)homopiperazine HCl (ML-9), H-Arg-Lys-Lys-Tyr-Lys-Tyr-Arg-Arg-Lys-NH₂ (MLCK inhibitor peptide 18), N-(6-aminohexyl)-1-naphthalenesulfonamide HCl (W-5), N-(6-aminohexyl)-5-chloro-1-naphthalenesulfonamide HCl (W-7), N-(4-aminobutyl)-2-naphthalenesulfonamide HCl (W-12), N-(4-aminobutyl)-5-chloro-2-naphthalenesulfonamide HCl (W-13), MLCK-expression inhibiting nucleotides such as targeted small interfering RNA (siRNA) knockdown agents against MLCK, anti-MLCK antibodies which may, for example be monoclonal or polyclonal, and inhibitory MLCK-binding fragments of such anti-MLCK antibodies such as Fab or F(ab)₂. Other salt forms or free base forms of the above-mentioned MLCK inhibitors may also be suitable.

Other suitable inhibitors of myosin activityation for use in the present invention include, for example, myosin phosphatase (MLCP) or its direct or indirect activators (MLCP activators) or direct or indirect inhibitors of its inhibitors (inhibitors of MLCP inhibitors). In the latter category there may be mentioned particularly direct or indirect inhibitors of CPI-17 (protein-kinase C-potentiated myosin phosphatase inhibitor, Mr=17 kDa), for example CPI-17-expression inhibiting nucleotides such as targeted siRNA knockdown agents against CPI-17, anti-CPI-17 antibodies which may, for example be monoclonal or polyclonal, and inhibitory CPI-17-binding fragments of such anti-CPI-17 antibodies such as Fab or F(ab)₂ (i.e. a direct CPI-17 inhibitor) or Rho kinase inhibitors and protein kinase C (PKC) inhibitors (i.e. indirect CPI-17 inhibitors). Examples of Rho kinase inhibitors that may be used in the present invention include 1-(1-hydroxy-5-isoquinolinesulfonyl)homopiperazine HCl (HA1100; Hydroxyfasudil), (S)-(+)-2-methyl-1-[(4-methyl-5-isoquinolinyl)sulfonyl]-homopiperazine 2HCl ROCK Inhibitor (H-1152; H-1152P; Rho kinase Inhibitor I), N-(4-pyridyl)-N′-(2,4,6-trichlorophenyl)urea (Rho kinase Inhibitor II), 3-(4-pyridyl)-1H-indole (Rho kinase Inhibitor III, Rockout), glycyl (S)-(+)-2-methyl-4-glycyl-1-(4-methylisoquinolinyl-5-sulfonyl)homopiperazine 2HCl (H-1152; Rho Kinase Inhibitor IV), (R)-(+)-trans-N-(4-pyridyl)-4-(1-aminoethyl)-cyclohexanecarboxamide 2HCl (Y27632 ROCK Inhibitor), Rho kinase-expression inhibiting nucleotides such as targeted siRNA knockdown agents against Rho kinase, anti-Rho kinase antibodies which may, for example, be monoclonal or polyclonal, and inhibitory Rho kinase-binding fragments of such anti-Rho kinase antibodies such as Fab or F(ab)₂. Examples of PKC inhibitors that may be used in the present invention include Myr-N-RKRTLRRL-OH myristoylated EGF-R fragment (651-658) Protein Kinase C inhibitor, Myr-N-FARKGALRQ-NH₂ myristoylated Protein Kinase C inhibitor 20-28 cell-permeable Protein Kinase C inhibitor, Protein Kinase C inhibitor peptide 19-36 RFARKGALRQKNVHEVKN, SIYRRGARRWRKL Protein Kinase C_{ζ/ι} pseudosubstrate inhibitor, Myr-SIYRRGARRWRKL-OH Protein Kinase C_{ζ} pseudosubstrate inhibitor myristoylated, LHQRRGAIKQAKVHHVKC-NH₂ Protein Kinase C_{θ} pseudosubstrate inhibitor, EAVSLKPT Protein Kinase C_{ε} translocation inhibitor peptide, RFARKGALRQKNV Protein Kinase C inhibitor peptide 19-31, Myr-LHQRRGAIKQAKVHHVKC-NH₂ Protein Kinase C_{θ} pseudosubstrate inhibitor myristoylated, PKC-expression inhibiting nucleotides such as targeted siRNA knockdown agents against PKC, anti-PKC antibodies which may, for example, be monoclonal or polyclonal, and inhibitory PKC-binding fragments of such anti-PKC antibodies such as Fab or F(ab)₂. Other salt forms or free base forms or other esterified forms of the above-mentioned Rho kinase and PKC inhibitors may also be suitable.

Antibody inhibitors may, for example, be polyclonal or monoclonal antibodies or any combination thereof. Inhibitory binding fragments of such antibodies may, for example, include Fab and F(ab)₂. Such antibodies/fragments are suitably prepared according to standard methods which need no detailed discussion here. Such inhibitors may be specific to more than one enzyme, provided that they have specificity for inhibition of at least one of the enzymes mentioned above, either directly or indirectly.

The peptide sequences of the inhibitors identified above, written with the amino terminus at the left and the carboxy terminus at the right and numbered in the conventional manner for sequences in patent documents, are as follows:

| | |
|---|---|
| Arg-Lys-Lys-Tyr-Lys-Tyr-Arg-Arg-Lys | SEQ. ID. NO: 1 |
| RKRTLRRL | SEQ. ID. NO: 2 |
| FARKGALRQ | SEQ. ID. NO: 3 |
| RFARKGALRQKNVHEVKN | SEQ. ID. NO: 4 |
| SIYRRGARRWRKL | SEQ. ID. NO: 5 |
| LHQRRGAIKQAKVHHVKC | SEQ. ID. NO: 6 |
| EAVSLKPT | SEQ. ID. NO: 7 |
| RFARKGALRQKNV | SEQ. ID. NO: 8 |

### Compositions and Administration Routes

The active agent and composition of the present invention may be administered by any suitable method, including (a) contacting the compositions (e.g. by topical administration or irrigation) with a target tissue or structure (i.e. a tissue or structure to which the active agent needs to be applied according to the present invention, for example a site at which fibrosis is to be prevented), or by administration by direct infusion or injection to a target tissue or structure, suitably during surgery, (b) slow release and/or slowly degradable compositions associated with a target tissue or structure or its vicinity, or (c) administration by direct infusion or injection to a target tissue or structure or its vicinity.

For example, in the treatment or prevention of fibrotic complications of glaucoma filtration surgery, the target tissue or structure may suitably be the sclera; in the treatment or prevention of fibrotic complications of cataract or other ocular lens replacement surgery, for example PCO, the target tissue or structure may suitably be the capsular bag; in the treatment or prevention of fibrotic complications of pterygia surgery, the target tissue or structure may suitably be the conjunctiva or cornea.

Conventional composition forms, such as aqueous or oily solutions or suspensions, gels, pastes, emulsions and sterile infusible or injectable aqueous or oily solutions or suspensions may be used.

For treatment or prevention of fibrotic complications of ocular lens replacement surgery, for example cataract surgery, the administration of the active agent and composition of the present invention is suitably by irrigation and/or by association with the lens and/or the capsular bag and/or its vicinity, or by administration by direct infusion or injection into the capsular bag and/or its vicinity.

Irrigation of the capsular bag during surgery (for example, cataract surgery involving lens replacement) according to the present invention may suitably be accomplished using a sealed-capsule irrigation device such as the PerfectCapsule® system (Milvella Pty. Ltd., Sydney, Australia) preferably before introduction of the new lens (see Abdelwahab et al, Journal of Cataract and Refractive Surgery, 33(9), 2007, 1619-1623). Such a device also provides a suitable means for introducing the active reagent and composition including as a slow release and/or slowly degradable composition in accordance with the present invention into the capsular bag Alternatively, or in addition, a slow release and/or slowly degradable composition in accordance with the present invention may be injected or infused into the capsular bag during the surgery.

Alternatively, or in addition, a slow release and/or slowly degradable composition in accordance with the present invention may be impregnated into, and/or coated onto, or otherwise associated with, the lens tissue of the new intraocular lens used in lens replacement surgery, so that the active agent will be gradually released into the capsular bag. The composition may be impregnated into, and/or coated onto, the lens in conventional manner, for example by soaking or spraying.

Thus, in a further aspect, the present invention provides an intraocular lens for use in ocular lens replacement surgery, wherein the lens has associated with it an active agent comprising an inhibitor of myosin activity, for the treatment or prevention of a fibrotic disorder of the lens, capsular bag, cornea, or other tissue or structure of the eye.

These composition forms will usually include one or more pharmaceutically acceptable ingredients which may be selected, for example, from adjuvants, carriers, binders, lubricants, diluents, sodium chloride, stabilising agents, buffering agents, emulsifying agents, wetting agents, viscosity-regulating agents, surfactants, preservatives, flavourings and colorants. As will be understood by those skilled in the art, the most appropriate method of administering the active ingredients is dependent on a number of factors. The compositions for use in the present invention may, if desired, be presented as a dry product for reconstitution with water or other suitable vehicle before use.

For control of release of the active agent according to the present invention a number of effective methods are available. See, for example, Wagh V. D., Inamdar B., Samanta M. K., Polymers used in ocular dosage form and drug delivery systems. Asian J Pharm 2, 2008, 12-17 and the literature references cited therein, the contents of which are incorporated herein by reference. The use of polymers (e.g. cellulose derivatives such as hydroxypropylmethylcellulose (HPMC) and hydroxypropylcellulose (HPC), poly(acrylic acid) (PAA), polyacrylates, cyclodextrins and natural gums, polyorthoesters (POEs) and mucoadhesive polymers, semisolids such as gels, films and other inserts, resins such as ion exchange resins, iontophoretic delivery, gene delivery via naked DNA or vector constructs, and colloidal particles such as microspheres and nanoparticles, may be particularly mentioned. Further discussion of each of these approaches is given in the Wagh paper cited above and the literature references therein, incorporated herein by reference.

In one embodiment of the present invention a number of ingredients are administered via separate pharmaceutical preparations. In this embodiment the different pharmaceutical preparations of active ingredients may be administered simultaneously, sequentially or separately.

Therefore, in one aspect, the present invention provides a kit comprising a preparation of a first active ingredient which is a myosin activity inhibitor, for example a specific or non-specific myosin activity inhibitor, and a preparation of a second active ingredient, and optionally instructions for the simultaneous, sequential or separate administration of the preparations to a patient in need thereof. Where sequential or separate administration of the preparations is carried out, the preparations may be administered in any order.

The second active ingredient may be selected from a wide range of physiologically active components, according to the likely or expected symptoms that may need to be relieved in the subject being treated. Such components may include, for example, an anti-inflammatory agent; a lipid lowering agent such as a statin or a fibrate; a modulator of blood cell morphology such as pentoxyfylline; a thrombolytic or an anticoagulant such as a platelet aggregation inhibitor; a CNS agent such as an antidepressant (such as sertraline); an agent for the treatment of acute or chronic pain, such as a centrally or peripherally-acting analgesic (for example an opioid or derivative thereof), carbamazepine, phenyloin, sodium valproate, amitryptiline or other anti-depressant agent(s), paracetamol, or a non-steroidal anti-inflammatory agent); a parenterally or topically-applied (including inhaled) local anaesthetic agent such as lignocaine or a derivative thereof; or any mixture or combination thereof.

The dosage of active agent to be applied is readily obtained by tests and establishing the dosage is within the skill of the reader, based on the experimental work described below. In any event, the dosage will need to be established according to the severity of the patient's symptoms, the age and weight of the patient, the susceptibility of the patient to adverse side effects and other factors normally considered in establishing a correct dose.

### BRIEF DESCRIPTION OF THE DRAWINGS

In the accompanying drawings:

FIG. 1 shows a diagrammatic representation of a post-surgical capsular bag and PCO development;

FIG. 2 shows the activation of myosin in the human lens epithelial cell line FHL124 in response to TGF-β;

FIG. 3 shows the suppression of TGF-β-induced matrix contraction by the myosin light chain kinase inhibitor ML7 using an FHL124 patch contraction assay;

FIG. 4 shows the promotion of TGF-β-induced matrix contraction by the myosin phosphatase inhibitor tautomycin using an FHL124 patch contraction assay;

FIG. 5 shows validation of siRNA directed against CPI-17.

FIG. 6 shows CPI-17 expression in response to TGF-β using immunocytochemistry;

FIG. 7 shows the inhibition of cell coverage across the posterior capsule of human lens capsular bags by ML7;

FIG. 8 shows cells growing on a lens capsular bag in response to TGF-β2, in the absence (left hand pair of photomicrographs, top being high power and bottom being low power) and presence (right hand pair of photomicrographs, top being high power and bottom being low power) of ML7 (note: matrix contraction/wrinkling was inhibited in the presence of ML7);

FIG. 9 shows the distribution of α-SMA, F-actin and nuclei (together in the bottom row of photomicrographs, α-SMA individualised in top row, F-actin individualised in the second row down, and nuclei individualised in the third row down) in cells growing on a lens capsular bag in response to TGF-β2, in the absence (left hand photomicrographs) and presence (right hand photomicrographs) of ML7.

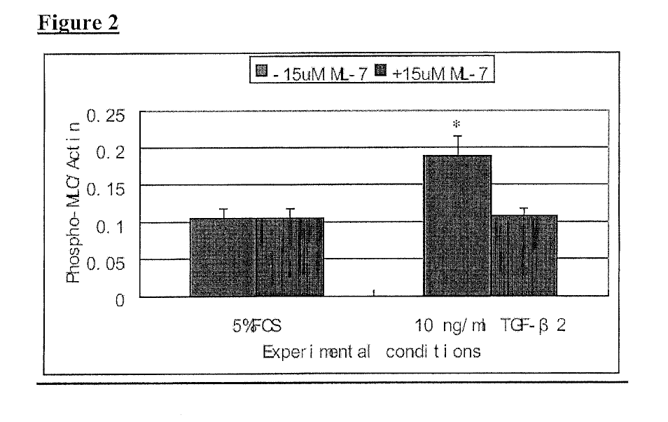

The following legends are helpful when studying FIGS. 2 to 9 of the drawings:

FIG. 2. Western blot detection of phosphorylated (active) myosin in response to 10 ng/ml TGF-β2 (Sigma, Poole, Dorset, UK). The data presented are pooled from 3 separate experiment. Each left hand bar of the bar pairs reports the result in the absence of ML7 (indicated as “−15 uM ML-7” in the key) and the right hand bar reports the result in the presence of 15 μM ML7 (indicated as “+15 uM ML-7” in the key). Beta actin was used as a control for equivalent protein amounts. * Indicates a significant difference from untreated control group (p<0.05; student's t-test).

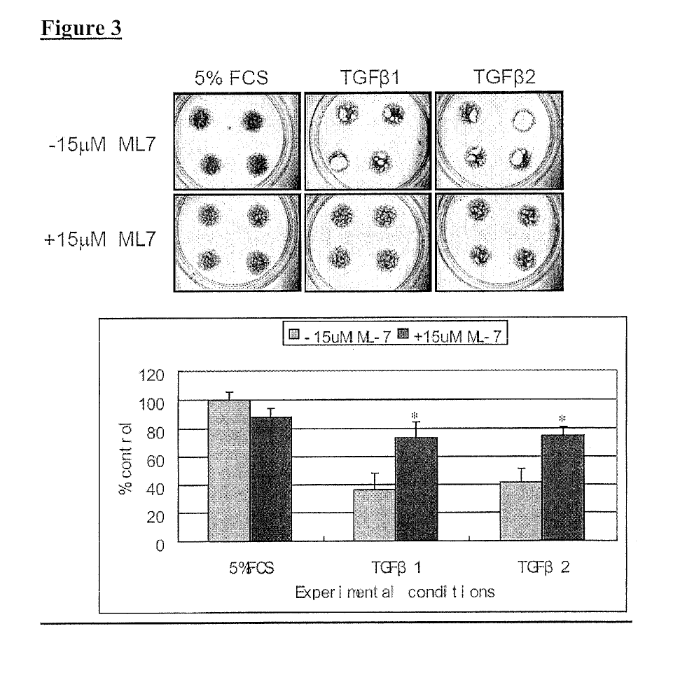

FIG. 3. TGF-β-induced matrix contraction is suppressed by the MLCK inhibitor ML7. FHL 124 cells were seeded to form patches and maintained in the following conditions: EMEM (Eagle's minimal essential medium—standard culture medium) supplemented with 5% FCS (fetal calf serum—control)±15 μM ML7 (myosin light chain kinase inhibitor) (Calbiochem) for a 72 hour experimental period. In the graph, each left hand bar of the bar pairs reports the result in the absence of ML7 (indicated as “−15 uM ML-7” in the key) and the right hand bar reports the result in the presence of 15 μM ML7 (indicated as “±15 uM ML-7” in the key). Please note that numerical data can be obtained from these patches, which can be scrutinized by statistical analysis. These experiments have been repeated on a number of occasions and in each case ML7 significantly suppresses TGF-β-induced contraction. * Indicates a significant difference from ML7 treated group relative to non-treated counterpart (p<0.05; student's t-test).

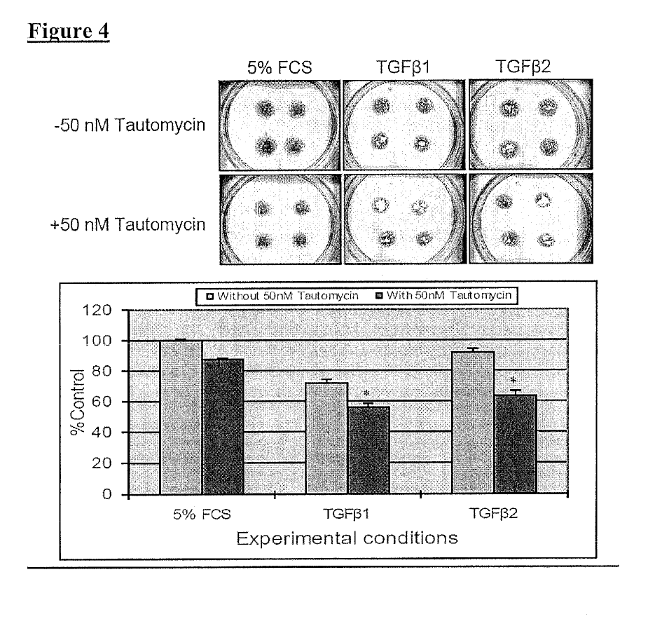

FIG. 4. TGF-β-induced matrix contraction is promoted by the MLCP inhibitor tautomycin (Calbiochem). FHL 124 cells were seeded to form patches and maintained in the following conditions: EMEM (standard culture medium) supplemented with 5% FCS (control)±50 nM tautomycin (myosin phosphatase inhibitor); control+10 ng/ml TGFβ1±50 nM tautomycin; control+10 ng/ml TGFβ2±50 nM tautomycin for a 48 hour experimental period. In the graph, each left hand bar of the bar pairs reports the result in the absence of tautomycin (indicated as “without 50 nM tautomycin” in the key) and the right hand bar reports the result in the presence of 50 nM tautomycin (indicated as “with 50 nM tautomycin” in the key). Please note that numerical data can be obtained from these patches, which can be scrutinized by statistical analysis. These experiments have been repeated on a number of occasions and in each case Tautomycin significantly increases TGF-β-induced contraction. * Indicates a significant difference from tautomycin treated group relative to non-treated counterpart (p<0.05; student's t-test).

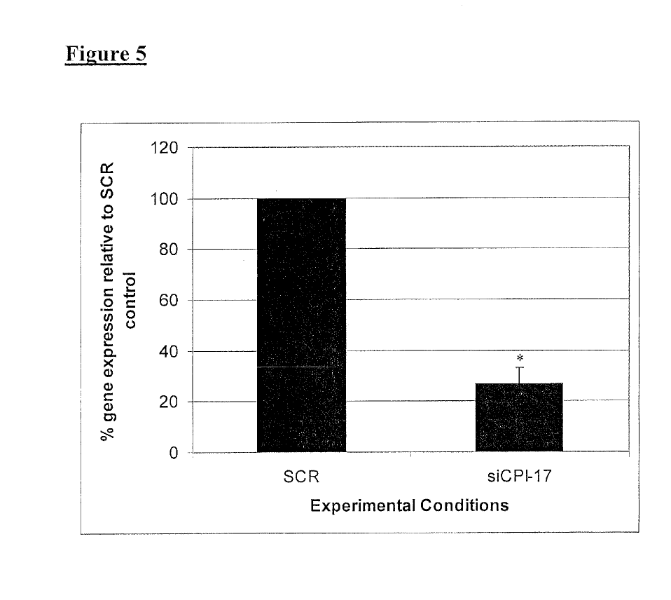

FIG. 5. Validation of siRNA directed against CPI-17. Quantitative real-time PCR (QRT-PCR) detection of CPI-17 gene expression in FHL 124 cells after 24 hours of transfection with siCPI-17 and negative siRNA control (SCR). Data were normalised with mGAPDH control and are expressed as the mean±SEM (n=4). *Significant difference between treated and untreated groups (P≦0.05, 1 tailed t-test).

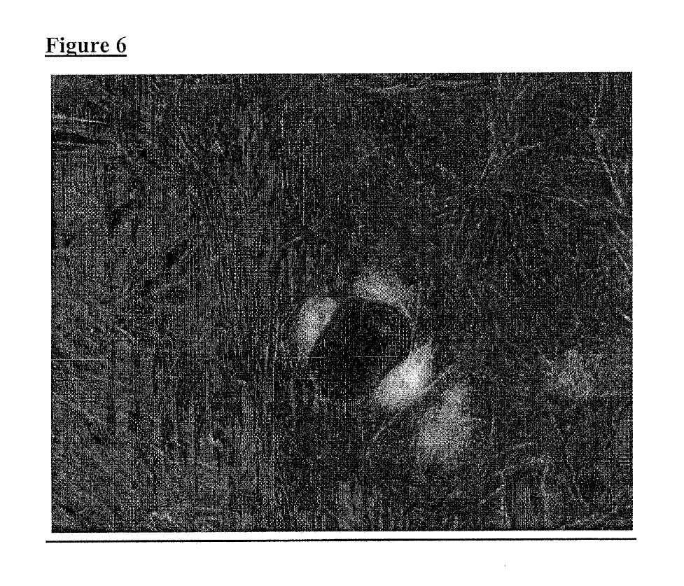

FIG. 6. A fluorescent micrograph showing CPI-17 expression in response to 10 ng/ml TGF-β. Intense expression of CPI-17 was observed adjacent to cell-free “holes”; these regions are likely to increase in size following a contractile event. The field of view represents 448×342 μm.

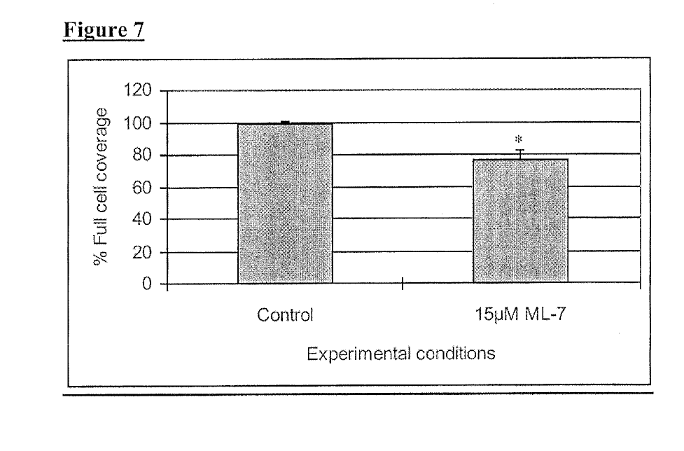

FIG. 7. The effect of MLCK inhibition (using 15 μM ML7) on cell coverage of the previously cell-free posterior capsule of human capsular bags following 7 days of culture. The data represent Mean SEM (n=4). * Indicates a significant difference from control group. N.B. Control conditions are EMEM supplemented with 5% serum and 10 ng/ml TGFβ.

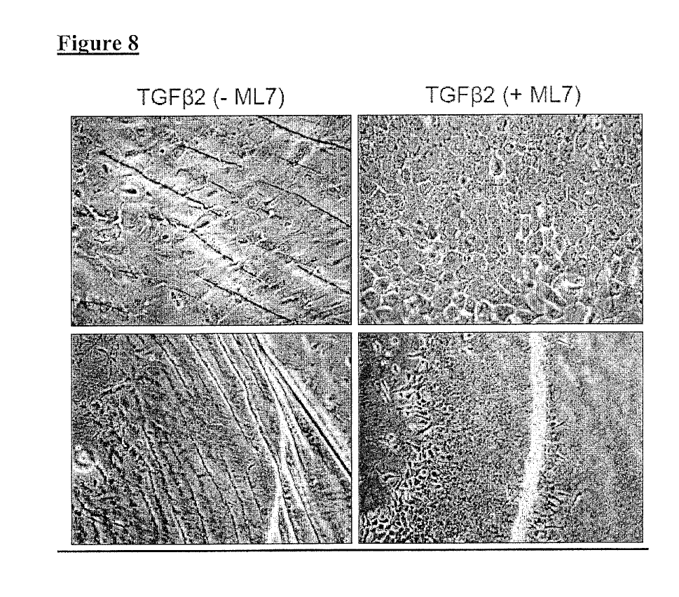

FIG. 8. High (top row) and low power (bottom row) phase-contrast micrographs showing cells growing on the lens capsular bag treated with 10 ng/mL TGF-β in the presence or absence of ML7 (15 μM). Contraction (wrinkles) of the lens capsular bag is evident when treated with 10 ng/ml TGF-β alone. However, treatment with TGF-β in the presence of ML7 does not give rise to wrinkle formation.

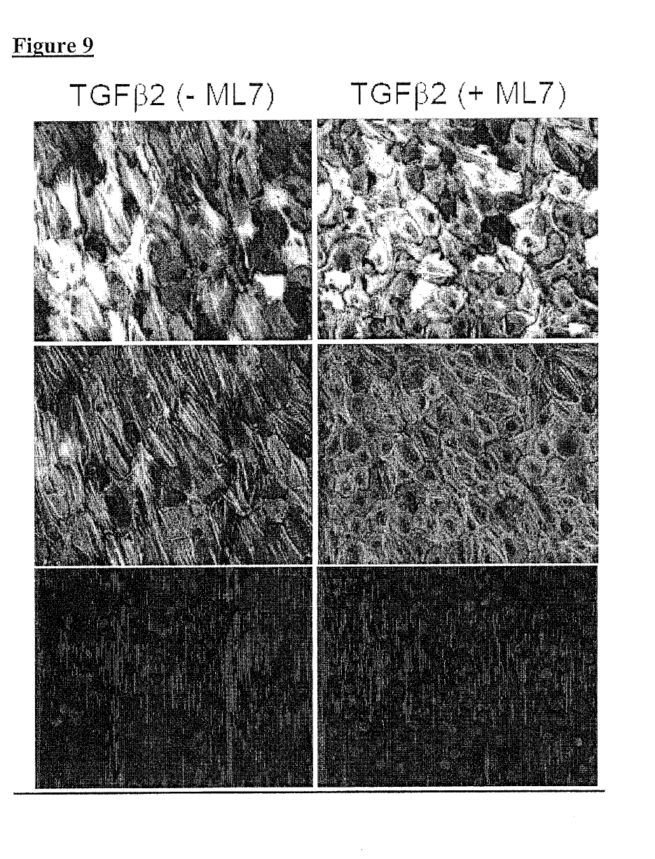

FIG. 9. Fluorescence micrographs showing distribution of α-smooth muscle actin, F-actin and nuclei (see the description of the Figure above, for identification of which photograph shows which features) in cells growing on the lens capsular bag treated with 10 ng/ml TGF-β in the presence or absence of ML7 (15 μM). The fields of view represent 448×342 μm.

### TEST DATA AND DETAILED DESCRIPTION OF THE DRAWINGS

The following non-limiting Test Data are provided for further illustration and explanation of the present invention and the accompanying drawings.

TGF-β is strongly linked with a number of pathologies including posterior capsule opacification (PCO), which develops in a significant number of patients following cataract surgery.

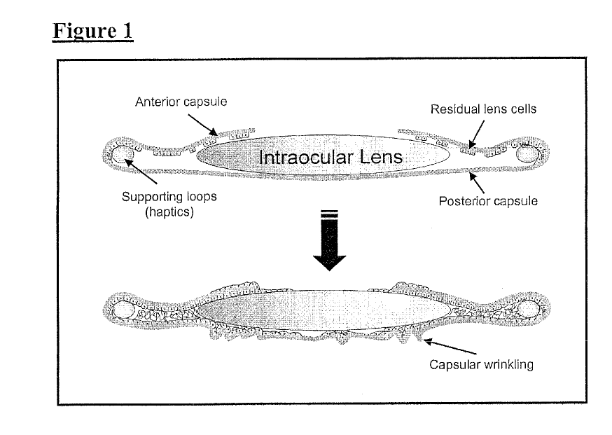

As illustrated diagrammatically in FIG. 1 of the drawings, in PCO, TGF-β causes the matrix on which the cells grow to deform through contraction. This is seen as wrinkling of the lens capsule. When this occurs on the central posterior capsule, the normal uninterrupted path of light becomes disrupted and no longer focuses properly on the retina. Consequently, this affects visual quality of a patient and can necessitate further corrective surgery.

The present invention is based on an important new understanding of the biological mechanisms mediating TGF-β-induced events, which in turn leads to therapeutic targets for the treatment of PCO and other TGF-β-associated conditions.

The data presented in the Figures and discussions here show that myosin regulation is critical for TGF-β-mediated matrix contraction. Myosin can exist in active (phosphorylated) and non-active states. Myosin light chain kinase (MLCK) can promote myosin activity while another protein, myosin phosphatase (MLCP), serves to reduce myosin activity. In addition, inhibition of another protein, CPI-17, can result in promotion of MLCP. The present invention provides treatment or prevention of fibrotic disorders by using these control systems for myosin activity to reduce matrix contraction through reduction in the overall level of active myosin,

### Materials and Methods

The materials and methods are as described above in the legends to each of FIGS. 2 to 9. FHL-124 cells were a gift from Dr John Reddan, Oakland University, Rochester, Mich., USA. TGF-β was sourced from Sigma-Aldrich (also referred to below as “Sigma”), Poole, Dorset, UK. ML7 and tautomycin were sourced from Calbiochem. siRNA was sourced from Ambion. Sources of other materials are stated below or the materials are easily available. Standard abbreviations are used for routine materials.

### Western Blot Analysis

FHL-124 cells were washed with 1.5 ml PBS then lysed on ice in 0.5 mls Daubs lysis buffer; (50 mM HEPES pH 7.5, 150 mM NaCl, 1% Triton X-100, 1 mM EDTA, 10% glycerol, 10 mM sodium pyrophosphate, 2 mM sodium orthovanadate, 10 mM sodium fluoride, 250 mls ddH₂O, 1 mM phenylmethylsulfonylfluoride and 10 μg/ml aprotinin. Lysates were pre-cleared by centrifuging at 13000 rpm at 4° C. for 10 min, and the protein content of the soluble fraction will be assayed by Bicinchoninic (BCA) protein assay. Equal amounts of protein per sample were loaded onto 10% SDS-PAGE gels for electrophoresis and transferred onto a polyvinylidene fluoride (PVDF) membrane with a BIO-RAD Trans-Blot semi-dry Transfer Cell. Primary antibodies for myosin (total and phosphorylated) and β-actin protein were incubated overnight and subsequently detected using the ECL plus blotting analysis system. Gels were scanned using an HP scanjet 5470c and band intensity determined with Kodak 1D 3.5 software.

### SiRNA Transfection

Custom made siRNA for the target gene (CPI-17) and siRNA negative control (universal scrambled siRNA), were used for this study and were purchased from Ambion Ltd, UK. FHL 124 cells were seeded onto 35 mm culture dishes at either 25,000 cells in 1.5 ml for protein extraction or as 4 patches of 5000 cells, for patch assay analysis. Cells were maintained in EMEM supplemented with 5% FCS for 3 days, and then serum starved for 1 day. Transfection was performed with 100 nM siRNAs according to the manufacturer's instructions. Briefly, 1 μl siRNA or siRNA negative control (final concentration of 100 nM) was added to 184 μl Optimem™ (Invitrogen Corporation; Optimem is a modified EMEM, buffered with HEPES and sodium bicarbonate and supplemented with hypoxanthine, thymidine, sodium pyruvate, L-glutamine or Glutamax, trace elements and growth factor; the protein level is minimal (15 μg/mL) with insulin and transferrin being the only protein supplements; phenol red is included as a pH indicator); 5 μl oligofectamine (Invitrogen) was added to 10 μl Optimem. The two separate solutions were then incubated at room temperature for 5 minutes and then mixed by gentle agitation and incubated at room temperature for a further 15 to 20 minutes. Meanwhile, the serum-free medium was aspirated from the cell preparations and replaced with 2 mls of Optimem. This solution was aspirated and replaced with 800 μl of fresh Optimem. Following the incubation period, 200 μl of siRNA transfection mix was added to the cell preparations. The cells were incubated at 35° C. in a 5% CO₂ atmosphere for 4 hours to initiate transfection. As the transfection procedure was continued for up to 48 hours, cell preparations were placed into experimental conditions after the four hour transfection period, with the addition of either 500 μl EMEM or 500 μl EMEM supplemented with 6% FCS, when prepared for cell lysis and patch assays respectively. Cells were lysed after 48 hours and patch assays terminated when notable contraction was observed.

### Patch Growth/Contraction Assay

FHL124 cells were seeded at 4 sites on a tissue culture dish at 5000 cells per site and maintained in EMEM supplemented with 5% FCS until confluent regions spanning approximately 5 mm developed. The medium was then replaced with non-supplemented EMEM and cells cultured for a further 24 hours. The cells were then exposed to experimental conditions and maintained for up to 3 days. Experiments were terminated by fixation for 30 minutes with 4% formaldehyde at room temperature. The cells were then washed in PBS and stained with Coomassie brilliant blue (a total protein dye) for 30 minutes to enable patches to be visualised and measured. The cells were then washed several times in PBS to remove excess dye. Images of patches were captured on a CCD camera using GeneSnap and analysed using Scion image. After the patch area was measured, PBS medium was aspirated from the culture dish and replaced with 1 ml of 70% ethanol allowing Coomassie blue dye within the cells to be dissolved. The culture dishes were placed on a rotary shaker for 1 hour until all the dye has been extracted from the cells. A 200 μl sample of dye from each dish was placed in a clear plastic 96 well microtitre plate and the absorbance read at 550 nm using a plate reader. This approach provided extra information as in addition to elucidating coverage and/or contraction relative population levels were established because total protein is reported to correlate with cell number (Knott, R. M., et al., Curr. Eye Res. (1998) 17, pages 1-8).

### Immunocytochemistry

Preparations were fixed for 30 minutes in 4% formaldehyde in PBS and permeabilised in PBS containing 0.5% Triton-X100, also for 30 minutes. Anti-CPI-17 and Anti-alpha smooth muscle actin was diluted 1:100 and applied for 60 min at 35° C., followed by washing. CPI-17 was visualised with ALEXA 488-conjugated secondary antibodies (Molecular Probes, Leiden, Netherlands). The F-actin cytoskeleton was stained with Texas Red-X-phalloidin and chromatin using DAPI (Molecular Probes, Leiden, Netherlands) for 10 min at room temperature. The stained preparations were again washed extensively, floated onto microscope slides and mounted in Hydromount mounting medium (National Diagnostics, Hull, UK). Images were viewed with a Zeiss epifluorescence microscope and Axiovision software.

### Human Lens Capsular Bag Model

The model first described by Liu, C. S. C., et al., (Invest. Ophthalmol. Vis. Sci. (1996), 37, pages, 906-914) was employed. Following removal of corneo-scleral discs for transplantation purposes, human donor eyes from the East Anglian Eye Banks, UK (obtained with full ethical approval—COREC form approved 1 Dec. 2004. Ref04/Q0102/57) were used to perform a sham cataract operation. The resulting capsular bag was then dissected free of the zonules and secured on a sterile 35 mm polymethylmethacrylate (PMMA) petri dish. Eight entomological pins were inserted through the edge of the capsule to retain its circular shape. Coverage and matrix modification of the previously cell-free posterior capsule were captured on a daily basis using low-power phase microscopy and quantified using analysis software.

### Results

### Myosin Phosphorylation

Application of 10 ng/ml TGF-β resulted in a significant increase in phosphorylated (active) myosin relative to non-stimulated controls (FIG. 2). Using the western blot method, it was shown that the MLCK inhibitor, ML7, could successfully inhibit myosin activation. Application of 15 μM ML7 significantly inhibited TGFβ-induced myosin activation (FIG. 2).

### Regulation of TGF-β Induced Matrix Contraction

We have selectively inhibited MLCK and MLCP with commercial agents, notably 15 μM ML7 (MLCK inhibitor) and 50 nM tautomycin (MLCP inhibitor) respectively. The addition of TGF-β to a circular monolayer of cells (a human lens cell line, FHL 124) results in contraction—this is visualized as “holes” (FIG. 3). These holes are devoid of cells and matrix (which the cells lay down in order to survive and grow). ML7 is an MLCK inhibitor and when applied to the cells causes a significant reduction of TGF-β-induced contraction (FIG. 3). In contrast, application of an MLCP inhibitor, tautomycin, speeded up the rate of contraction (FIG. 4).

Another molecule we investigated was CPI-17 ((protein-kinase C-potentiated myosin phosphatase inhibitor; Mr=17 kDa)). This particular protein is regulated by a signalling system known as the Rho kinase pathway. When this pathway is stimulated, CPI-17 becomes active and is believed to inhibit the actions of MLCP.

We therefore set out to detect CPI-17 in the human lens cells and determine its involvement in TGFβ-mediated contraction. We have achieved some of our objectives in this case, but not all. In the first instance, through use of real-time PCR and western blots we have demonstrated that CPI-17 is expressed by the lens cells. (FIG. 5). Moreover, we then tested whether siRNA methods can be used to prevent CPI-17 being expressed in lens cells; siRNA is a relatively new molecular method that specifically targets a gene. SiRNA targeted against CPI-17 was purchased from a commercial supplier and successful inhibition at the message level has been achieved (FIG. 5).

We have also studied the distribution of CPI-17 in response to TGF-β using immunocytochemistry and fluorescence microscopy. Cells treated with TGF-β demonstrated an interesting distribution pattern, such that there was a high expression adjacent to small cell free “holes” (FIG. 6). One can reasonably predict that these holes would increase in size due to TGF-β-induced contraction. This therefore provides a link between TGF-β, CPI-17 and matrix contraction, which we believe will be confirmed by other tests such as inhibition studies.

The experiments above were carried out on a human lens cell line (FHL 124), but it is important to be able to translate this information to predictions and results for the clinic. To achieve this objective, we used a human capsular bag model. As described above, this method involves carrying out a sham cataract operation on human donor eyes—the product of the operation is known as a capsular bag. The capsular bags, which can be cultured and studied in the laboratory (in vitro) has the same organization as the bag in the patient (in vivo) and therefore serves as the best predictor of real-life events that is currently available.

From the work on the cell line we chose to test ML7 i.e. the MLCK inhibitor. These studies were carried out on separate donors using a match paired format; one capsular bag was maintained in TGF-β and serum supplemented medium, while the other was maintained in TGF-β and serum supplemented medium plus 15 μM ML7. There is great consistency in cell behaviour from one eye to another from an individual donor; therefore the effects observed are due to the presence or absence of the inhibitor. To begin with, we observed an inhibition of growth across the previously cell-free posterior capsule, such that 80% of the posterior capsule was covered with cells in the ML7 treated group, whereas 100% cover was observed in the control group (FIG. 7). This growth inhibition could be useful in the treatment of PCO as growth across the capsule is a critical component in the development of the condition, but this is a retardation of cell growth rather than ablation. Perhaps more importantly, MLCK inhibition using ML7 can suppress TGF-β-induced matrix contraction (this time seen as wrinkles; FIG. 8). In addition, we have carried out immunocytochemical analysis of the capsular bags at end point (FIG. 9). This work shows that the organization of cells treated with ML7 is different to untreated controls.

### Discussion and Conclusions

Traditionally, TGF-β was believed to cause contraction due to a transformation of the cells from an epithelial cell type to a myofibroblast cell type. This latter type of cells express a protein called alpha smooth muscle actin (αSMA), which was thought to give rise to contraction. We have recently shown (Dawes et al., Invest. Ophthalmol. Vis. Sci. (February 2008), 49(2), pages 650-661) that αSMA is not responsible for contraction to occur. The data reveal that αSMA levels in both ML7 treated and control bags are similar, thus providing extra support for this notion. Interestingly, the general organization of the cells is different following ML7 treatment. In control TGF-β/serum bags, the cells appear more dynamic and actin, a cytoskeleton protein, exhibits bright stress filaments; following ML7 treatment the cells exhibit a cobblestone appearance. Additionally, in the ML7 group there is an even distribution of cell nuclei whereas the control bags show orientation along cell wrinkles (sites of contraction) and different planes of focus, which suggests multilayering.

Promotion of myosin activity by MLCK is associated with increased matrix contraction, while the inhibitory actions of MLCP serve to decrease contraction. Myosin activity in the presence of TGF-β can be reduced by the MLCK inhibitor ML7 or promoted by the MLCP inhibitor tautomycin; this pattern correlates to matrix contraction. Thus, it can be concluded that regulation of myosin activity by TGF-β is integral to matrix contraction. Consequently, application of agents that can reduce the activity of myosin via these regulatory pathways may thus be used to develop novel therapeutic treatments to prevent/inhibit visual loss associated with fibrotic disorders of the eye, for example PCO.

The foregoing describes the present invention broadly and without limitation to particular embodiments. Variations and modifications as will be readily apparent to those skilled in the art are intended to be included in the scope of the application and subsequent patents.

### SEQUENCE LISTING

| | |
|---|---|
| Arg-Lys-Lys-Tyr-Lys-Tyr-Arg-Arg-Lys | SEQ. ID. NO: 1 |
| RKRTLRRL | SEQ. ID. NO: 2 |
| FARKGALRQ | SEQ. ID. NO: 3 |
| RFARKGALRQKNVHEVKN | SEQ. ID. NO: 4 |
| SIYRRGARRWRKL | SEQ. ID. NO: 5 |
| LHQRRGAIKQAKVHHVKC | SEQ. ID. NO: 6 |
| EAVSLKPT | SEQ. ID. NO: 7 |
| RFARKGALRQKNV | SEQ. ID. NO: 8 |

All artificial protein sequences.

1. Use, in a method for the treatment or prevention of a fibrotic disorder of the eye, or in the preparation of a medicament therefor, of an active agent comprising an inhibitor of myosin activity.

2. An active agent comprising an inhibitor of myosin activity, for use in the treatment or prevention of a fibrotic disorder of the eye.

3. A composition, for example a pharmaceutical composition (medicament), comprising an effective amount of an active agent comprising an inhibitor of myosin activity, and one or more physiologically compatible carrier, diluent or excipient, for use in the treatment or prevention of a fibrotic disorder of the eye.

4. An agent according to claim 2, wherein the myosin activity inhibitor is a specific inhibitor of myosin activity and is used at a concentration where the said inhibition is specific to myosin.

5. An agent according to claim 2, wherein the myosin activity inhibitor is a non-specific inhibitor of myosin activity.

6. An agent according to claim 2, wherein the myosin activity inhibitor comprises a MLCK inhibitor.

7. An agent according to claim 6, wherein the MLCK inhibitor is selected from ML7, (5-iodonaphthalene-1-sulfonyl)homopiperazine (ML7), 1-(5-chloronaphthalene-1-sulfonyl)homopiperazine HCl (ML-9), H-Arg-Lys-Lys-Tyr-Lys-Tyr-Arg-Arg-Lys-NH₂ (SEQ. ID. NO: 1) (MLCK inhibitor peptide 18), N-(6-aminohexyl)-1-naphthalenesulfonamide HCl (W-5), N-(6-aminohexyl)-5-chloro-1-naphthalenesulfonamide HCl (W-7), N-(4-aminobutyl)-2-naphthalenesulfonamide HCl (W-12), N-(4-aminobutyl)-5-chloro-2-naphthalenesulfonamide HCl (W-13), targeted small interfering RNA (siRNA) knockdown agents against MLCK, anti-MLCK antibodies, and inhibitory MLCK-binding fragments of such anti-MLCK antibodies.

8. An agent according to claim 2, wherein the myosin activity inhibitor comprises, or further comprises, myosin phosphatase (MLCP) or a direct or indirect activator thereof (MLCP activator) or a direct or indirect inhibitor of an inhibitor thereof (inhibitor of an MLCP inhibitor).

9. An agent according to claim 8, wherein the myosin activity inhibitor is an inhibitor of CPI-17 (protein-kinase C-potentiated myosin phosphatase inhibitor, Mr=17 kDa).

10. An agent according to claim 8, wherein the CPI-17 inhibitor is selected from targeted siRNA knockdown agents against CPI-17, anti-CPI-17 antibodies, inhibitory CPI-17-binding fragments of such anti-CPI-17 antibodies, Rho kinase inhibitors and protein kinase C (PKC) inhibitors.

11. An agent according to claim 10, wherein the Rho kinase inhibitor is selected from 1-(1-hydroxy-5-isoquinolinesulfonyl)homopiperazine HCl (HA 1100; Hydroxyfasudil), (S)-(+)-2-methyl-1-[(4-methyl-5-isoquinolinyl)sulfonyl]-homopiperazine 2HCl ROCK Inhibitor (H-1152; H-1152P; Rho kinase Inhibitor I), N-(4-pyridyl)-N′-(2,4,6-trichlorophenyl)urea (Rho kinase Inhibitor II), 3-(4-pyridyl)-1H-indole (Rho kinase Inhibitor III, Rockout), glycyl (S)-(+)-2-methyl-4-glycyl-1-(4-methylisoquinolinyl-5-sulfonyl)homopiperazine 2HCl (H-1152; Rho Kinase Inhibitor IV), (R)-(+)-trans-N-(4-pyridyl)-4-(1-aminoethyl)-cyclohexanecarboxamide 2HCl (Y27632 ROCK Inhibitor), targeted siRNA knockdown agents against Rho kinase, anti-Rho kinase antibodies, and inhibitory Rho kinase-binding fragments of such anti-Rho kinase antibodies.

12. An agent or according to claim 10, wherein the PKC inhibitor is selected from Myr-N-RKRTLRRL-OH (SEQ. ID. NO: 2) myristoylated EGF-R fragment (651-658) Protein Kinase C inhibitor, Myr-N-FARKGALRQ-NH₂ (SEQ. ID. NO: 3) myristoylated Protein Kinase C inhibitor 20-28 cell-permeable Protein Kinase C inhibitor, Protein Kinase C inhibitor peptide 19-36 RFARKGALRQKNVHEVKN (SEQ. ID. NO: 4), SIYRRGARRWRKL (SEQ. ID. NO: 5) Protein Kinase C_{ζ/ι} pseudosubstrate inhibitor, Myr-SIYRRGARRWRKL-OH (SEQ. ID. NO: 5) Protein Kinase C_{ζ} pseudosubstrate inhibitor myristoylated, LHQRRGAIKQAKVHHVKC-NH₂ (SEQ. ID. NO: 6) Protein Kinase C_{θ} pseudosubstrate inhibitor, EAVSLKPT (SEQ. ID. NO: 7) Protein Kinase C_{ζ} translocation inhibitor peptide, RFARKGALRQKNV (SEQ. ID. NO: 8) Protein Kinase C inhibitor peptide 19-31, Myr-LHQRRGAIKQAKVHHVKC-NH₂ (SEQ. ID. NO: 6) Protein Kinase C_{θ} pseudosubstrate inhibitor myristoylated, targeted siRNA knockdown agents against PKC, anti-PKC antibodies, and inhibitory PKC-binding fragments of such anti-PKC antibodies.

13. A composition according to claim 3, which is a slow release and/or slowly degradable composition.

14. A composition according to claim 3, for the treatment or prevention of a fibrotic disorder of the eye by (a) contacting the compositions (e.g. by topical administration or irrigation) with a target tissue or structure (i.e. a tissue or structure to which the active agent needs to be applied according to the present invention, for example a site at which fibrosis is to be prevented), or by administration by direct infusion or injection to a target tissue or structure, suitably during surgery, (b) slow release and/or slowly degradable compositions associated with a target tissue or structure or its vicinity, or (c) administration by direct infusion or injection to a target tissue or structure or its vicinity.

15. A composition according to claim 14, wherein: for treatment or prevention of fibrotic complications of glaucoma filtration surgery, the target tissue or structure is the sclera; for treatment or prevention of fibrotic complications of cataract surgery, for example PCO, the target tissue or structure is the capsular bag; or for treatment or prevention of fibrotic complications of pterygia surgery, the target tissue or structure is the conjunctiva or cornea.

16. A composition according to claim 3, for use in ocular lens replacement surgery by irrigation and/or by association with the lens and/or the capsular bag and/or its vicinity, or by administration by direct infusion or injection into the capsular bag and/or its vicinity.

17. An intraocular lens for use in ocular lens replacement surgery, wherein the lens has associated with it an active agent comprising an inhibitor of myosin activity, for the treatment or prevention of a fibrotic disorder of the lens, capsular bag, cornea, or other tissue or structure of the eye.

18. A lens according to claim 17, wherein the myosin activity inhibitor is a specific inhibitor of myosin activity and is used at a concentration where the inhibition is specific to myosin or is present in a composition comprising an effective amount of an active agent comprising an inhibitor of myosin activity, and one or more physiologically compatible carrier, diluent or excipient.

19. A kit for use in the treatment or prevention of a fibrotic disorder of the eye comprising a preparation of a first active ingredient which is a myosin activity inhibitor and a preparation of a second active ingredient, and optionally instructions for the simultaneous, sequential or separate administration of the preparations to a patient in need thereof.

20. A kit according to claim 19, wherein the myosin activity inhibitor is a specific inhibitor of myosin activity and is used at a concentration where the said inhibition is specific to myosin or is present in a composition comprising an effective amount of an active agent comprising an inhibitor of myosin activity, and one or more physiologically compatible carrier, diluent or excipient.

21. A lens according to claim 17, wherein the fibrotic disorder of the eye is a TGF-β-mediated disorder.

22. A lens according to claim 17, wherein the fibrotic disorder of the eye is PCO or other complication of cataract surgery.

23. A lens according to claim 17, wherein the fibrotic disorder of the eye is a complication of glaucoma surgery.

24. A lens according to claim 17, wherein the fibrotic disorder of the eye is a complication of pterygia surgery.


SEQUENCE LISTING (8 sequences)

SEQ ID NO: 1 - protein, 9 aa, Artificial
  MLCK inhibitor peptide 18
RKKYKYRRK

SEQ ID NO: 2 - protein, 8 aa, Artificial
  myristoylated EGF-R fragment (651-658) Protein Kinase C inhibitor
RKRTLRRL

SEQ ID NO: 3 - protein, 9 aa, Artificial
  myristoylated Protein Kinase C inhibitor 20-28 cell permeable Protein Kinase C inhibitor
FARKGALRQ

SEQ ID NO: 4 - protein, 18 aa, Artificial
  Protein Kinase C inhibitor peptide 19-36
RFARKGALRQKNVHEVKN

SEQ ID NO: 5 - protein, 13 aa, Artificial
  Protein Kinase C-zeta/lota pseudosubstrate inhibitor; or Protein Kinase C-zeta  pseudosubstrate inhibitor myristoylated
SIYRRGARRWRKL

SEQ ID NO: 6 - protein, 18 aa, Artificial
  Protein Kinase C-theta pseudosubstrate inhibitor; or Protein Kinase C-theta pseudosubstrate inhibitor myristoylated
LHQRRGAIKQAKVHHVKC

SEQ ID NO: 7 - protein, 8 aa, Artificial
  Protein Kinase C-epsilon translocation inhibitor peptide
EAVSLKPT

SEQ ID NO: 8 - protein, 13 aa, Artificial
  Protein Kinase C inhibitor peptide 19-31
RFARKGALRQKNV